(19) United States
(12) Patent Application Publication
(10) Pub. No.: US 2009/0208368 A1
(43) Pub. Date: Aug. 20, 2009

(54) URINARY CATHETER, CATHETER PACKAGING ASSEMBLY AND METHOD OF USE

(76) Inventors: Kent Waldrep, Celina, TX (US); Danny R. Cox, Celina, TX (US); Hector Ladron Barra, Jalisco (MX)

Correspondence Address: WHITAKER, CHALK, SWINDLE & SAWYER, LLP, 3500 CITY CENTER TOWER II, 301 COMMERCE STREET, FORT WORTH, TX, 76102-4186, US

(21) Appl. No.: 12/031,252

(22) Filed: Feb. 14, 2008

(51) Int. Cl. A61L 2/18; B65D 25/08

(52) U.S. Cl. 422/28; 206/219

(57) ABSTRACT

A urinary catheter and companion packaging system for packaging sterile items utilized during a catheterization procedure are shown. A lightweight, flexible package contains a collection of ancillary materials and a catheter package. The catheter package houses a solution pouch, a protection sheath, and a catheter within the protection sheath. A saline solution is contained within the solution pouch, and is capable of being released from the solution pouch once external pressure breaks a weakened seam along one side of the solution pouch. A hydrophilic coating on the catheter activates once the saline solution contacts the exterior surface of the catheter. The catheter has an insertion tip that has been oven blasted and rounded in order to reduce the coefficient of friction. The overall length of the catheter is greater than about sixteen inches.

### BACKGROUND OF THE INVENTION

1. Field of the Invention

The present invention provides, in combination, a urinary catheter and a companion packaging system for packaging the catheter along with sterile items utilized during a catheterization procedure.

2. Description of the Prior Art

According to the usual medical definition, a catheter is a tube that is inserted into a body cavity, duct or vessel to thereby allow the drainage of fluids. More specifically, a urinary catheter is a plastic tube that is inserted into a patient's urinary tract and into their bladder in order to drain urine for a variety of medical purposes.

Polyvinyl chloride (PVC) has become the medical industry standard for use in manufacturing urinary catheters. PVC possesses several key characteristics that satisfy the requirements needed for catheter use. Namely, PVC is soft, exhibits kink resistance, and possesses a surface that is easily sterilized or exposed to other surface treatments. A typical catheter design of the type under consideration can be seen in U.S. Pat. No. 5,919,170.

During catheterization, whether it be a medical procedure or a self-catheterization, there are oftentimes several additional items required. As a result, it is useful to package such items together, to further assist in a speedy catheterization. U.S. Pat. No. 4,811,847 teaches a rectangular tray with a pull-back top containing a catheter assembly, a syringe with bacteriostatic water, and two gloves. However, this design is rather large and bulky, and a series of such packages would possibly prove difficult for an individual to transport.

It is generally necessary or at least desirable that catheters be lubricated prior to insertion into the patient's body. In earlier times, catheters would be removed from a sterile package and manually lubricated. However, this procedure may prove time-consuming and also risk the sterility of the catheter. U.S. Pat. No. 3,967,728 teaches a catheter package that includes a rupturable, lubricant containing pouch inside the package and adjacent the tip of the catheter. The pouch contains sterile lubricant, and the portion of the pouch which is adjacent to the tip of the catheter will fail when the pouch is squeezed, thus emptying lubricant onto the catheter tip.

A more recent development is shown in U.S. Pat. No. 6,709,706 which describes a medical device intended for introduction into the human body, the device being coated with a hydrophilic coating which becomes lubricous when contacted by an aqueous fluid.

Despite improvements of the above type, a need exists therefore to provide a catheter, having further reduced-friction characteristics.

Existing urinary catheters presently available in the marketplace have generally been provided in overall lengths of about sixteen inches or less. It would be desirable to provide a urinary catheter having a greater overall length. A length on the order of twenty four inches overall would be more convenient to use, for example in allowing a patient to access a toilet, either at home or in a hospital.

A need also exists to provide a packaging system for all required items necessary for a catheterization procedure, which packaging system is lightweight and flexible.

A need also exists for such a packaging system to provide a quick and simple method for ensuring sterilization of the catheter, while providing easy access to the catheter for use.

### SUMMARY OF THE INVENTION

It is the general object of the present invention to provide, in combination, a urinary catheter and companion packaging system for packaging sterile items utilized during a catheterization procedure. The combination is housed within a lightweight outer package, which has a flexible exterior. The exterior defines a watertight interior space therein, wherein the interior space is adapted for storing a collection of sterilized ancillary materials together with a primary catheter package. The collection of ancillary materials may include, for example, a set of examination gloves, lubricating jelly, one or more preparation pads, a drape, a supply of gauze, and a collection bag. The preparation pads can include, for example, both a povidone iodine preparation pad and a benzalkonium chloride preparation pad.

The primary catheter package comprises an elongated body having an exterior and an enclosed interior, a length, a top region and a bottom region. The top region of the primary package is provided with at least one finger opening which also forms a point for suspending the package from a nail or hook. In addition, the primary catheter package is further provided with a plurality of weakened seams which form tear regions along the length thereof.

A solution pouch and a protective sheath are housed within the interior space of the primary catheter package proximate the top region thereof. The solution pouch has an exterior surface and an interior surface, defined between a first and second extent thereof. A saline solution is contained within the interior of the solution pouch. The solution pouch is provided with a weakened seam along the length thereof generally from the first extent to the second extent. The pouch weakened seam is capable of failing upon the application of external pressure to the exterior of the solution pouch.

A catheter is located within the interior of the primary catheter package and extends generally along the length thereof within the protective sheath. The catheter has an outer diameter and an inner diameter and a length defined between a first and second extent thereof. The catheter first extent forms an insertion tip having a plurality of apertures therein, and the catheter second extent terminates in a flexible, cup-shaped discharge region. The catheter first extent which forms the insertion tip is specially heat treated by being oven blasted and rounded in order to provide a tip having a reduced coefficient of friction. The catheter can be formed of polyvinyl chloride (PVC). The catheter is preferably between about 17 and 24 inches in length. Furthermore, the catheter has a hydrophilic coating that is activated by contact with the contents of the solution pouch.

A method is also provided for preparing for a catheterization procedure utilizing the sterile items from a prepackaged system of the type described above. To begin, the outer package is opened and the ancillary materials and primary catheter package are extracted. Next, the saline solution is released into the primary catheter package by applying external pressure to the exterior of the solution pouch. The primary catheter package is then hung by the hollow opening in the top region to allow the released saline solution to flow along the catheter from the top region to the bottom region of the primary catheter package. Lastly, the primary catheter package is torn along the tear regions to create an opening. The catheter may then be removed from the catheter package while pushing the protection sheath along the catheter to the insertion tip.

Additional objects, features and advantages will be apparent in the written description which follows.

### BRIEF DESCRIPTION OF THE DRAWINGS

FIG. 1 is a perspective view of the outer package of the packaging system of the present invention.

FIG. 2 is an exploded view of the packaging system of FIG. 1 showing the contents thereof.

FIG. 3A is an enlarged, partial view of the insertion tip of the catheter of the present invention.

FIG. 3B is an enlarged, partial view of the drainage valve end of the catheter of the present invention.

FIG. 4 is an enlarged view of the primary catheter package found in the packaging system of FIG. 2.

FIG. 5 is an exploded perspective view of the solution pouch found in the primary catheter package of FIG. 4.

### DETAILED DESCRIPTION OF THE INVENTION

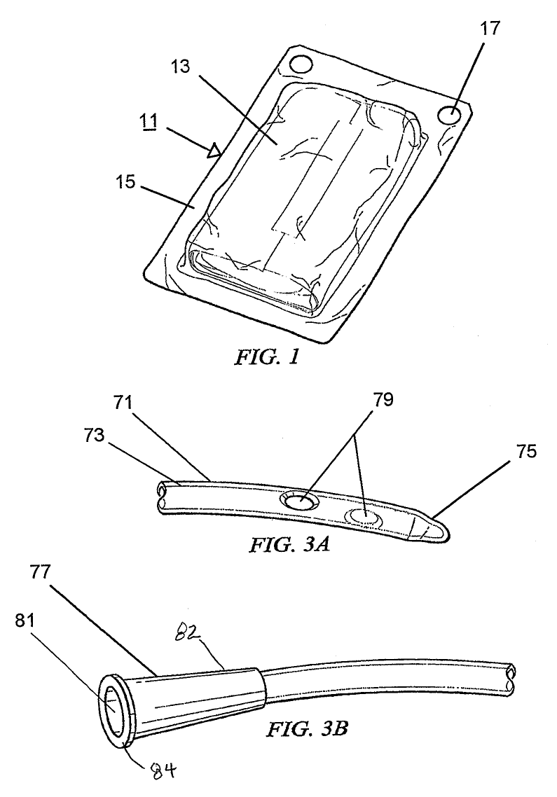

Turning to FIGS. 1-5, there is shown the preferred embodiment of the present invention. It is the general object of the present invention to provide, in combination, an improved catheter and companion packaging system for the catheter for packaging sterile items utilized during a catheterization procedure.

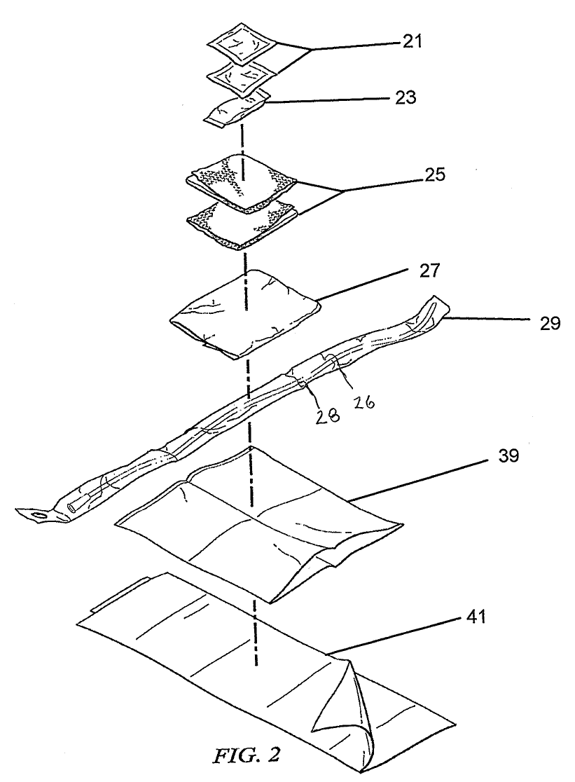

The combination urinary catheter and sterile items are housed within a lightweight outer package 11, as seen in FIG. 1, which has a flexible exterior 15. It is crucial that the outer package 11 be lightweight, as it is sometimes necessary for individuals to carry multiple packages at a time. This can be accomplished by forming the outer package of a suitable lightweight plastic, such as a suitable polyolefin. The flexible nature of the outer package 11 allow a varied range of storage locations. For instance, individuals who employ the use of a wheelchair may place the package behind their back, or sit atop, the flexible outer package 11 without causing discomfort or harm. The exterior 15 of the outer package defines a watertight interior space 13 therein. The interior space 13 is adapted for storing a collection of sterilized ancillary materials together with a primary catheter package. FIG. 2 provides a perspective view of each component found within the outer package. The collection of ancillary materials can include, for example, a set of examination gloves 27, lubricating jelly 23, one or more preparation pads 21, a drape 41, a supply of gauze 25, and a collection bag 39. The preparation pads 21 preferably comprise a povidone iodine preparation pad and a benzalkonium chloride preparation pad, both of which are commonly used as antiseptics.

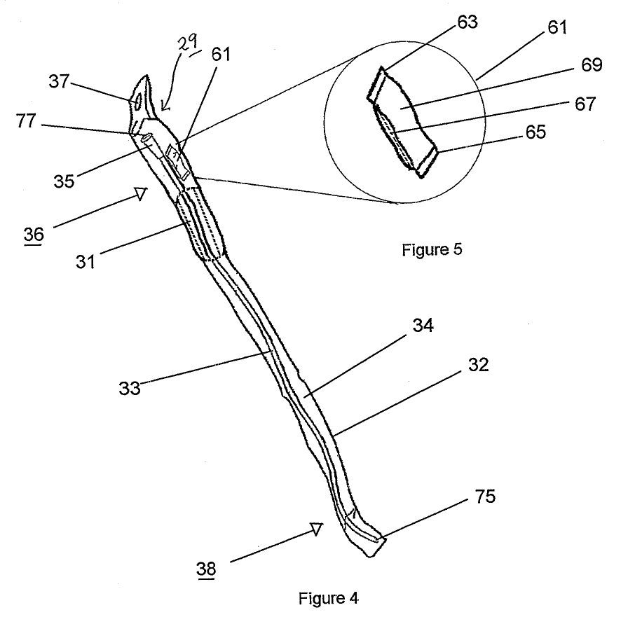

The primary catheter package 29 comprises an elongated body having an exterior 32 and an enclosed interior 34, a length, a top region and a bottom region (generally shown as 36 and 38 in FIG. 4, respectively). The top region 36 of the primary package 29 is provided with at least one finger opening 37. In addition, the primary catheter package 29 is further provided with a plurality of weakened seams such as seams 26, 28 in FIG. 2, which form tear regions along the length thereof. A solution pouch 61 and a protective sheath 31 (see FIG. 4) are housed within the interior space 34 of the primary catheter package 29 proximate the top region 36 thereof. FIG. 5 illustrates an expanded view of the solution pouch 61 found in the top region 36 of the catheter package 29. The solution pouch 61 has an exterior surface 69 and an interior surface, defined between a first and second extent (63 and 65, respectively) thereof. A saline solution is contained within the interior of the solution pouch 61. The solution pouch is provided with a weakened seam 67 along the length thereof generally from the first extent 63 to the second extent 65. The pouch weakened seam 67 is capable of failing upon the application of external pressure to the exterior 69 of the solution pouch 61.

Turning to FIG. 4, an expanded view of the primary catheter package 29 is shown containing the protective sheath 31 and the catheter 33. As can be seen in FIG. 4, the catheter 33 is located within the interior of the primary catheter package 29 and extends generally along the length thereof within the protective sheath 31. The catheter 33 has an outer diameter 71 and an inner diameter 73 (as shown in FIG. 3A) and a length defined between a first and second extent thereof (75 and 77, respectively). FIG. 3A provides an exploded view of the catheter first extent 75, which forms an insertion tip having a plurality of apertures 79 therein. Likewise, FIG. 3B provides an exploded view of the catheter second extent 77 which terminates in a flexible, cup-shaped discharge region 81.

The catheter first extent 75, which forms the insertion tip, is specially heat treated by being oven blasted and rounded in order to provide a tip having a reduced coefficient of friction. The flexible, cup-shaped discharge region 81 directs urine is a simple current flow and prevents the urine from spraying upon exiting the catheter. As can be seen FIG. 3B, the cup-shaped discharge region 81 has gently sloping, outwardly flowing sidewalls 82. The discharge region 81 terminates in a circular-shaped end ring 84 which circumscribes the end opening of the catheter. As has been mentioned, the catheter can be formed of any suitable plastic, and in this case is formed of polyvinyl chloride (PVC). The catheter 33 is preferably between 17 and 24 inches in length, leading to a greater flexibility as opposed to prior catheter designs, and producing greater ease of use due to the increased length. Furthermore, the catheter has a hydrophilic coating that is activated by contact with the contents of the solution pouch. The hydrophilic nature of the catheter coating helps to insure the flexibility of the catheter and provides reduced-friction characteristics.

A method is also provided for preparing for a catheterization procedure utilizing the sterile items from a prepackaged system of the type described above. Once the prepackaged system has been provided, the outer package is opened and the ancillary materials and catheter package are extracted. Next, the saline solution is released into the catheter package by applying external pressure to the exterior of the solution pouch 61. The catheter package is then hung by the hollow opening 37 to allow the released saline solution to flow along the catheter from the top region to the bottom region of the catheter package. Lastly, the catheter package is torn along the tear regions 26, 28 to create an opening. The catheter may then be removed from the catheter package while pushing the protection sheath along the catheter to the insertion tip.

An invention has been provided with several advantages. The combination urinary catheter and companion packaging system provides a convenient, lightweight, flexible and sterile collection of all necessary items needed for a catheterization procedure. In addition, the present catheter, with oven blasted and rounded tip design as well as hydrophilic coating, provides a further friction-reducing characteristics. The cup-shaped discharge region reduces any tendency to spray during use.

While the invention has been shown in only one of its forms, it is not thus limited but is susceptible to various changes and modifications without departing from the spirit thereof.

I claim:

1. In combination, a urinary catheter and companion packaging system for packaging sterile items utilized during a catheterization procedure, the combination comprising:
a lightweight outer package having a flexible exterior which defines a watertight interior space therein, the interior space being adapted for storing a collection of sterilized ancillary materials together with a primary catheter package;
wherein the primary catheter package comprises an elongated body having an exterior and an enclosed interior, a length, a top region and a bottom region, the top region of the primary package being provided with at least one finger opening, and wherein the primary catheter package is further provided with a plurality of weakened seams which form tear regions along the length thereof;
wherein a solution pouch and a protection sheath are housed within the interior space of the primary catheter package proximate the top region thereof, the solution pouch having an exterior surface and an interior surface, defined between a first and second extent thereof, and wherein a saline solution is contained within the interior of the solution pouch, the solution pouch also being provided with a weakened seam along the length thereof generally from the first extent to the second extent, the pouch weakened seam being capable of failing upon the application of external pressure to the exterior of the solution pouch; and
a catheter located within the interior of the primary catheter package and extending generally along the length thereof within the protective sheath, the catheter having an outer diameter and an inner diameter and a length defined between a first and second extent thereof, the catheter first extent forming an insertion tip having a plurality of apertures therein, said catheter second extend terminating in a flexible, cup-shaped discharge region.

2. The combination catheter and packaging system of claim 1, wherein the collection of ancillary materials includes a set of examination gloves, lubricating jelly, one or more preparation pads, a drape, a supply of gauze, and a collection bag.

3. The combination catheter and packaging system of claim 2, wherein the preparation pads include both a povidone iodine preparation pad and a benzalkonium chloride preparation pad.

4. The combination catheter and packaging system of claim 1, wherein said catheter is formed of polyvinyl chloride (PVC).

5. The combination catheter and packaging system of claim 4, wherein said catheter has a hydrophilic coating that is activated by contact with contents of the solution pouch.

6. The combination catheter and packaging system of claim 1, wherein said catheter is between 17 and 24 inches in length.

7. The combination catheter and packaging system of claim 1, wherein the catheter first extent which forms the insertion tip is specially heat treated by being oven blasted and rounded in order to provide a tip having a reduced coefficient of friction.

8. A method of preparing for a catheterization procedure utilizing the sterile items from a prepackaged system, the steps of the method comprising:
providing a lightweight outer package having a flexible exterior which defines a watertight interior space therein, wherein a collection of sterilized ancillary materials and a catheter package are housed within said interior space therein;
wherein the catheter package is defined by an elongated and narrow exterior which encloses a watertight interior space, said catheter package having a length, a top region, and a bottom region, and wherein there is a hollow opening at said top region, and wherein there are a plurality of tear regions along the length of the catheter package defined by a weakened seam;
providing a solution pouch and a protection sheath housed within said interior space of the catheter package, the solution pouch located at the top region of the catheter package and having an exterior and interior surface with a first and second extent, providing a saline solution contained within the interior of said solution pouch, and wherein there is a weakened seam along the length of the exterior surface from the first extent to the second extent capable of breaking failing;
providing a catheter housed along the length of the catheter package within the protection sheath, said catheter having an outer diameter and an inner diameter, a length terminating in a first and second extent, said first extent having an insertion tip with a plurality of hollow bores and said second extent having a drainage valve;
opening the outer package and extracting the ancillary materials and catheter package;
releasing the saline solution into the catheter package by applying external pressure to the exterior of the solution pouch;
hanging the catheter package by the hollow opening and allowing the released saline solution to flow along the catheter from the top region to the bottom region of the catheter package; and
tearing the catheter package along the tear region to create an opening and removing the catheter from the catheter package while pushing the protection sheath along the catheter to the insertion tip.
